(19) United States
(12) Patent Application Publication
(10) Pub. No.: US 2011/0053874 A1
(43) Pub. Date: Mar. 3, 2011

(54) USE OF FLAVONOIDE COMPOUNDS FOR THE PROPHYLAXIS AND THERAPY OF ISCHAEMIC OR INFLAMMATORY HEART AND CARDIOVASCULAR DISEASES

(75) Inventors: Anke Esperester, Mainz (DE); Stephan Nees, Munchen (DE)

(73) Assignee: BOEHRINGER INGELHEIM INTERNATIONAL GMBH, Ingelheim am Rhein (DE)

(21) Appl. No.: 12/919,561

(22) PCT Filed: Mar. 3, 2009
(86) PCT No.: PCT/EP09/52502

§ 371 Date: Nov. 12, 2010

(30) Foreign Application Priority Data
Mar. 6, 2008 (DE) 102008012909.7

(51) Int. Cl. A61K 31/7048; A61P 9/04; A61P 9/10

(52) U.S. Cl. 514/27

(57) ABSTRACT

The present invention relates to the use of flavonoid compounds, particularly quercetin-3-O-β-D-glucuronide and kaempferol-3-O-β-D-glucuronide and their glucosides, for preventing diseases of the heart. Red vine leaf extract has proved a particularly advantageous source of the flavonoid compounds in question.

### SCOPE OF THE INVENTION

The present invention relates to the use of flavonoid compounds, particularly flavonol glucosides and flavonol glucuronides, especially quercetin-3-O-β-D-glucoside, kaempferol-3-O-β-D-glucoside and the corresponding glucuronides, quercetin-3-O-β-D-glucuronide and kaempferol-3-O-β-D-glucuronide, for preventing heart and circulatory disease. Certain vegetable extracts, e.g. endive, celery or red vine leaf extract have proved a particularly advantageous source of the flavonol compounds in question.

### BACKGROUND TO THE INVENTION

Ischaemically induced functional disorders of organs are still the main cause of death in all industrialised countries, primary myocardial infarct and, ever more frequently, septically induced perfusion disorders. In the last 10-15 years the advantageous effects of extensive consumption of fruit and vegetables on heart health and particularly on the risk of myocardial infarct have repeatedly been demonstrated in epidemiological studies (Yusuf et al., Lancet 2004; 364(9438):937-952; Knekt et al., BMJ 1996; 312:478-481; Feng et al., Lancet 2006; 367:320-326). A current Cochrane Review comprising 23 studies came to the conclusion that the consumption of vegetables has a positive influence on heart health. The consumption of fruit on its own, on the other hand, did not appear to have a significant effect on heart health (Brunnet et al., Cochrane Database of Systematic Reviews 2005, Issue 4, Art. No.:CD002128).

Usually the studies in question do not mention the type of fruit or vegetable eaten. Therefore it appears to be difficult to lay down criteria that ensure that nutrition has a positive effect on heart health. Thus, polyphenols have been proposed as an important factor. However, the class of polyphenols include an extensive palette of sub-groups such as catechols, flavonoids, procyanidines or isoflavones, for example. All these sub-groups have been credited with many cell- or organ-protective effects on the basis of in vitro studies. Corresponding plant material which is rich in polyphenols and has been extracted from the plant is brought into direct contact with cell or organ systems. However, in vivo the target organs are not brought into contact with the substances in the same way. For one thing, a number of polyphenols are hardly absorbed at all in the digestive tract. Moreover, the absorbed substances are metabolised (Manach et al. Am J Clin Nutr 2005; 81(1 Suppl):230S-242S). Therefore it is not clear from in vitro studies whether a sufficiently high concentration of a substance actually reaches the bloodstream or whether the substance then present in the bloodstream is still physiologically relevant (Manach et al. Am J Clin Nutr 2005; 81(1 Suppl):230S-242S; Kroon et al. Am J Clin Nutr 2004; 80: 15-21).

Quercetin is one of the most investigated flavonols, both in vitro and after parenteral administration to animals (Formica et al., Fd Chem. Toxic 1995; 12:1061-1080). However, quercetin is only absorbed to a small extent (Manach et al. Am J Clin Nutr 2005; 81(1 Suppl):230S-242S). Quercetin occurs in nature in the form of glycosides and glucuronides (McAnlis et al., Eur J Clin Nutr 1999; 53(2):92-96; Graefe et al., Journal of Clinical Pharmacology 2001; 41(5):492-499). It has been found that the corresponding flavonol glucosides are absorbed to some extent and occur in the plasma in the form of the respective glucuronides. Little is known of the in vivo effects of these compounds. In particular, there have not as yet been any studies into a protective effect of flavonol glucuronides on the human heart. There is doubt as to whether quercetin glucuronide could have an effect on LDL oxidation, which is an important mechanism within the scope of heart failure (McAnlis et al., Eur J Clin Nutr 1999; 53(2):92-96).

### SUMMARY OF THE INVENTION

It has now been found that flavonoids, especially flavonol glucuronides, in particular quercetin-3-0-β-D-glucuronide and kaempferol-3-0-β-D-glucuronide, have a cardioprotective effect. The likelihood of myocardial infarct and heart failure in particular can be reduced by administering these substances.

Flavonol glucuronides are poorly absorbed in the gastrointestinal tract. Therefore, in previous medical applications (e.g. in the treatment of CVI) their corresponding glucosides have been used as prodrugs. Red vine leaf extract is particularly rich in quercetin glucoside and kaempferol glucoside. Human kinetic data are available which demonstrate that the two compounds after being taken orally and absorbed through the intestine are present in the plasma almost completely in the form of their respective glucuronides. Oral administration produces plasma concentrations which are regarded as sufficient for prophylactic protection from cardiac and circulatory disorders. In the case of acute ischaemic or inflammation-induced and life-threatening organ function disorders, the active substances prepared in pure form are best not taken orally but rather intravasally, most preferably intraarterially.

In order to improve or prevent ischaemic or inflammatory processes or conditions the present invention therefore also comprises intraarterial administration and use of purely prepared quercetin-3-O-β-D-glucuronide and/or kaempferol-3-O-β-D-glucuronide for reducing the risk of functional organ failure, which may occur under analogous conditions in all organs and not just in the heart. In addition, the use of quercetin glucoside and/or kaempferol glucoside on their own or as an extract from red vine leaf, which has a high concentration of both compounds, as a prodrug for oral administration is also claimed.

### DETAILED DESCRIPTION OF THE INVENTION

“Flavonol compounds” in the sense of the present invention are substances with a 3-hydroxyflavone structure, particularly those with free hydroxyl groups. Preferred flavonols are derivatives of quercetin and kaempferol. Particularly preferred flavonol compounds within the scope of the present invention are quercetin glucuronide and kaempferol glucuronide, particularly quercetin-3-O-β-D-glucuronide and kaempferol-3-O-β-D-glucuronide.

Within the scope of the present invention there are various possible administration routes. On the one hand, flavonoid compounds, preferably quercetin-3-O-β-D-glucuronide and kaempferol-3-O-β-D-glucuronide, may be administered by intravascular, including intraarterial route. This would also get round the problem of low absorption through the digestive tract.

However, intravasal administration of this kind is of only limited value for routine preventive measures.

Alternatively, the flavonol compounds may also be administered by oral route. For this purpose, apart from the corresponding pure glucosides, which are then metabolised as “prodrugs” to form the corresponding glucuronides as described hereinbefore, it is possible to use in particular plant extracts which are rich in flavonoids. Particularly advantageous in this context are red vine leaf extracts which have a high content of flavonoids. Particularly preferred is the red vine leaf extract described in EP 1 225 810, which may be obtained both as an aqueous extract and as a dry extract. This extract is particularly rich in quercetin-3-O-β-D-glucoside and kaempferol-3-O-β-D-glucoside and may be administered either as drops or as capsules or tablets. The extract mentioned in EP 1 225 810 contains 2 to 20% flavonoids, while the aqueous red vine leaf extract may be obtained by a process comprising the following steps:
(a) collecting red vine leaves at a time when the flavonoid content reaches an optimum level;
(b) drying and shredding the leaves;
(c) cutting the leaves into pieces;
(d) extracting the leaves with water at temperatures of 60 to 80° C. for 6 to 10 hours by total percolation;
(e) optionally concentrating the resulting extract.

An extract thus obtained is marketed under the name AS195 by Boehringer Ingelheim.

This extract is suitable for oral administration, particularly in a solid dosage form, i.e. as a capsule or tablet which is made up of 20 to 60% of an aqueous extract of red vine leaves with a high content of flavonoids of 2-15%. Another preferred dosage form consists of drops containing 3 to 90% of the extract. Other suitable administration forms may include coated tablets, syrups or the like. The extract is characterised by a high content of 2 to 20% and preferably 2 to 10% of biologically active flavonoids.

Carriers or excipients may be added during the drying in order to make the further processing of the extract easier. Such carriers or excipients may be silicon dioxide, maltodextrin, glucose syrup, cellulose and the like.

Preferred forms for administration are tablets, including coated tablets or capsules. However, liquid preparations, preferably drops, may also be chosen.

A preferred alternative embodiment of an orally administered preparation in the sense of the present invention is a film-coated tablet, particularly as proposed in EP 1 581 195.

The film-coated tablet disclosed therein contains 50 to 70% of a dry extract of red vine leaf with a flavonoid content of 2-15%, produced by the aqueous extraction method described above.

The film-coated tablet also contains excipients in the tablet core. The weight ratio of extract to the excipients is from 1:1 to 2:1, preferably from 1.1:1 to 1.8:1, preferably from 1.25: 1 to 1.75: 1.

A film-coated tablet by way of example contains
(a) 50 to 70 wt. % dry extract of red vine leaf;
(b) 25 to 49 wt. % excipients, and
(c) 1 to 5 wt. % film coating, based on the total mass of the film-coated tablet.

In an embodiment by way of example the excipients (b) consist of
70 to 85 wt. % of a binder,
0.5 to 12.5 wt. % of a disintegrant,
5 to 15 wt. % of a filler, and
1 to 5 wt. % of a flow agent and lubricant,
   based on the total mass of the excipients.

“Binder” denotes an excipient that binds the ingredients to one another. Preferred binders are: cellulose powder, microcrystalline cellulose, sorbitol, starch, povidone, copolymers of vinylpyrrolidone with other derivatives (copovidone), cellulose derivatives, particularly methylhydroxypropylcellulose, e.g. Methocel A 15 LV, and mixtures thereof. The preferred binders are cellulose powder, microcrystalline cellulose and/or povidone. The above-mentioned binders are used in a range of 15-45 wt. %, preferably 25-40 wt. %, preferably 33 wt. %, measured against the total weight of the tablet.

The tablet according to the invention also contains disintegrants in addition to the ingredients mentioned above. These are preferably selected from among sodium starch glycolate, crospovidone, croscarmellose sodium salt (sodium salt of cellulose carboxymethylether, crosslinked), sodium-carboxymethylcellulose, dried maize starch, colloidal anhydrous silica and mixtures thereof. The above-mentioned disintegrants are used in a range of 0.5-10 wt. %, preferably 1.5-7.5 wt. %, measured against the total weight of the tablet.

The tablet according to the invention also comprises a filler which is described as an inert inorganic metal oxide or phosphate or hydrogen phosphate. Calcium hydrogen phosphate is the preferred filler. The above-mentioned fillers are used in a range of 1-10 wt. %, preferably 2-8 wt. %, measured against the total weight of the tablet.

The tablet according to the invention also contains flow agents and/or lubricants in addition to the ingredients mentioned above. These include silicon dioxide, talc, stearic acid, sodium stearylfumarate, magnesium stearate and glycerol tribehenate. The above-mentioned flow agents and lubricants are used in a range of 0.1-10 wt. %, preferably 0.6 and 1.5 wt. %, measured against the total weight of the tablet.

Other suitable sources of flavonols are vegetable extracts, e.g. extracts of endive, celery, etc.

The following is a description of some experimental investigations in which the activity of flavonols in ischaemic states was considered.

### Preservation Procedure for Guinea-Pig Hearts

Female guinea pigs (250-330 g) were used as heart donors. After the animals were decapitated their hearts were explanted and placed in a Langendorff apparatus (specially constructed). Perfusion was carried out retrogressively through the aorta under normal conditions under a constant pressure of 60 mmHg for 3 min (mode 1). Krebs-Henseleit-bicarbonate buffer (KHM) which was gassed with carbogen before use was used for the perfusion, without added quercetin glucuronide (QG), at a temperature of 37° C. After cannulation of the left atrium, the apparatus was switched to operating mode (mode 2) with a preload of 10 mmHg and an afterload of 60 mmHg. After 2 minutes' perfusion the basic functions were recorded, specifically: aortic flow, coronary flow, ejection rate, heart rate, maximum systolic pressure, mean arterial pressure, and the product of the heart rate and maximum systolic pressure. Then the apparatus was switched to mode 1, and the hearts, divided into 2 groups, were perfused further with HTK solution chilled to a temperature of 4° C. (=Bretschneider's cardioprotective solution), with or without the addition of 100 μM QG, until the heart stopped in each case, and then stored in the dark at 4° C. in the same perfusion medium (30 ml in each case) for a period of 8 h. Then the hearts were placed in the Langendorff apparatus once again and perfused under normal conditions in mode 1. Finally, the apparatus was switched back into operating mode and the performance data defined above were measured under these conditions as well. Result: All the values measured in the hearts preserved with the addition of QG during the 8 hour ischaemic period were 25-35% above those of the comparison hearts (no QG added to the HTK solution).

### Preservation Procedure for Human Heart Apices

Human hearts from patients with a known blood group, explanted in the course of heart transplants, are used as the starting material for the preparation of perfusible heart apices. A circumfusion with 100 ml of the plasma substitute solution “Biseko” (made by Biotest, Dreieich), in which the total calcium had previously been adjusted to 2.5 mM, is carried out for 30 minutes at 37° C. through cannulas tied into the transected epicardial coronary arteries. This procedure is repeated with another 100 ml aliquot of adjusted and warmed Biseko solution. Then a mixture of 96 ml of Ca-substituted Biseko and 4 ml of a supernatant (sedimented at high speed) of a suspension of highly concentrated neutrophilic granulocytes (PMN) and thrombocytes (T) (about 10⁶ or 10⁷/ml) previously activated by the addition of 100 μM ADP and 1 μM FMLP is circumfused through the coronary system of the heart apices for 10 min at 37° C., or—in another test group—100 μM QG are also added to the circumfusion fluid. Then 50 ml of whole blood of the same group are added to the circumfusion fluid, the PMN and T being activated immediately beforehand by the addition of FMLP and ADP (analogous concentration to that mentioned above), or 100 μM of QG are also present—in the 2nd test group. This is followed by 45 minutes of circumfusion at 37° C. Then Evans Blue is added until a clear blue coloration is obtained, after another 3 minutes' circumfusion the blue stained areas of the heart apices are dissected out, cut up into small cubes (with sides 5 mm long) and frozen in liquid nitrogen. Histological sections (30 μm thick) are prepared from the frozen tissue and arterioles or venules in the myocardial tissue are identified by their typical marker enzymes (alkaline phosphatase or dipeptidylamino peptidase). The PMN are identified with monoclonal anti-PMN-antibody (MBL, Japan), the T are identified immunohistologically with monoclonal anti-CD61 and counted under the microscope. Result: Whereas in the absence of QG massive clots of platelets are formed particularly in the region of the coronary arterioles and adhesion and diapedesis of the PMN occurs selectively in the venules, pathophysiological interactions of the two types of blood cell with the coronary vascular system cannot be detected in the presence of the flavonoid.

1. A method of using quercetin-3-O-β-D-glucuronide, kaempferol-3-O-β-D-glucuronide, or a combination thereof for preventing diseases of the heart induced ischaemically or by inflammation.

2. The method according to claim 1, wherein the quercetin-3-O-β-D-glucuronide, and/or kaempferol-3-O-β-D-glucuronide, or the combination thereof is administered by intravascular route.

3. The method according to claim 1, wherein the quercetin-3-O-β-D-glucuronide, kaempferol-3-O-β-D-glucuronide, or the combination thereof is administered by oral route in the form of their corresponding glucosides.

4. The method according to claim 1, wherein quercetin-3-O-β-D-glucoside or kaempferol-3-O-β-D-glucoside is administered by oral route in the form of their corresponding glucosides.

5. The method according to claim 3, wherein the quercetin-3-O-β-D-glucuronide, kaempferol-3-O-β-D-glucuronide, or the combination thereof is part of a composition.

6. The method according to claim 5, wherein the composition is an extract of red vine leaf.

7. The method according to claim 6, wherein the extract used is the one marketed as AS195.

8. The method according to claim 1, wherein the diseases of the heart are selected from among myocardial infarct and heart failure.
